(19) United States
(12) Patent Application Publication
(10) Pub. No.: US 2021/0137453 A1
(43) Pub. Date: May 13, 2021

(54) SYSTEMS AND METHODS FOR SELF-GUIDED INJURY TREATMENT

(71) Applicant: MD ORTHO SYSTEMS LLC, Earlysville, VA (US)

(72) Inventor: Thomas S. Weber, Earlysville, VA (US)

(21) Appl. No.: 17/096,131

(22) Filed: Nov. 12, 2020

Related U.S. Application Data
(60) Provisional application No. 62/934,057, filed on Nov. 12, 2019.

(51) Int. Cl. A61B 5/00; G16H 20/00; G16H 50/20

(52) U.S. Cl. CPC A61B 5/4836; A61B 5/7475; G16H 50/20; G16H 20/00

(57) ABSTRACT

The present disclosure relates to systems and methods for self-guided treatment of deformities, disorders, or injuries of the musculoskeletal system. In particular, the disclosure relates to user-interfacing systems and methods of using same for selection of an appropriate medical equipment and or treatment plan for a user.

### PRIORITY STATEMENT

This application claims priority to U.S. Provisional Application No. 62/934,057, filed on Nov. 12, 2019, the entire contents of which are incorporated herein by reference.

### FIELD

The present disclosure relates to systems and methods for self-guided treatment of deformities, disorders, or injuries of the musculoskeletal system. In particular, the disclosure relates to user-interfacing systems and methods of using same for selection of an appropriate medical equipment and or treatment plan for a user.

### BACKGROUND

Hundreds of millions of Americans and people worldwide seek treatment for orthopedic conditions annually. With the population continuing to age, even more will seek care on their own apart from the traditional locations they have used over the past 100 years in healthcare. Local and rapid access to care are common roadblocks that prevent patients from getting appropriate advice or examinations for their conditions. With the advent of the internet and cloud-based resources, medical websites often give people a place to start or recommendations on treating health conditions prior to seeing their healthcare provider.

However, when it comes to orthopedic braces, which are often sold over the counter and without a prescription, patient are often guessing as to which brace is right for their injury or pain or symptoms. With trends in healthcare moving to an outpatient world, the need for “self-service” options is critical to meet the demands of a growing population with both age related degenerative orthopedic conditions and younger, active populations with sports related or exercise related joint conditions and injuries.

### BRIEF SUMMARY

The present disclosure relates to systems and methods for self-guided treatment of deformities, disorders, or injuries of the skeleton and associated structures such as tendons and ligaments, and methods of using the systems.

In one aspect, provided herein are systems for self-guided treatment of deformities, disorders, or injuries of the skeleton and associated structures. In some embodiments, the systems include a graphical user interface and a processor. In some embodiments, the systems include a graphical user interface, a processor, and computer-readable code. The graphical user interface is configured to receive data regarding a deformity, disorder, and/or injury from a user and transmit the data to the processor. For example, the graphical user interface may be configured to receive data regarding a deformity, disorder, and/or injury to the skeleton and/or associated structure from a user and transmit the data to the processor. In some embodiments, the processor is configured to analyze the data and provide output to the user. In some embodiments, the computer-readable code is configured to instruct the processor to analyze (e.g., apply an algorithm to) the data. The disclosure is not limited to any particular way of analyzing (e.g., applying an algorithm to) the data, or to the type of information generated upon analysis (e.g., provided by the algorithm to the user or to another aspect of the system).

In one embodiment, analyzing the data comprises applying an algorithm to the data to determine and/or to identify an appropriate medical equipment for the deformity, disorder, and/or injury; however, the disclosure is not so limited. In another embodiment, analyzing the data comprises applying an algorithm to the data to generate and provide information to the user related to the identification of the deformity, disorder, and/or injury, and/or provide information related to a treatment (e.g., a treatment plan) for the deformity, disorder, and/or injury. In some embodiments, analyzing the data comprises applying an algorithm to the data to generate and provide information to the user related to the identification (e.g., a differential diagnosis) of the deformity, disorder, and/or injury and recommend a treatment plan (e.g., an orthopedic brace, exercise, rest, or combination of same) for the deformity, disorder, and/or injury.

In other embodiments, analyzing the data comprises applying an algorithm to the data to generate and provide information to the user about actions the user may take regarding the deformity, disorder, and/or injury. The disclosure is not limited to any particular information provided to the user related to actions the user may take. Indeed, a variety of information may be provided to the user including actions such as, but not limited to, instructions pertaining to obtaining a scan (e.g., x-ray, magnetic resonance image (MRI), computed tomography (CT or CAT), ultrasound, electromyogram, or other type of scan) of the deformity, disorder, or injury, instructions to make an appointment with a physician, instructions to call 9-1-1, and/or instructions regarding care for the deformity, disorder, or injury. In some embodiments, instructions regarding care comprise identification of a brace for the deformity, disorder, and/or injury. In other embodiments, instructions regarding care comprise recommended actions for the user to take. The disclosure is not limited by the type of action recommended. Non-limiting examples of recommended actions include rest, ice, heat, immobilization (e.g., using a brace or wrap), exercise, therapy, stretching, strengthening, to seek the opinion of an expert (e.g., an in-person or virtual visit with a physician), seek immediate emergency care, and obtain a scan (e.g., x-ray, magnetic resonance image (MRI), computed tomography (CT or CAT), ultrasound, or other type of scan).

In some embodiments, the system further comprises a database of service providers. The system may be configured to apply the data provided by the user recommend a local service provider or a list of local service providers to the user. For example, the system may maintain a database of service providers (e.g. physicians including available telehealth physicians, clinics, pharmacies, urgent care facilities, emergency rooms, etc.) that may be accessed by the system to determine the appropriate recommendation for the user. In some embodiments, the system may additionally maintain information regarding diagnostic capabilities of the service providers (e.g. MRI capability, X-ray capability, etc.) to ensure that the service provider recommended to the user is capable of performing the necessary diagnostic procedure and/or treatment. For example, the system may maintain a list of service providers from which to draw from to provide the user with a recommendation for a visit with a service provider to evaluate the user, have a telehealth visit with the user, perform a diagnostic imaging procedure on the user, provide physical therapy to the user, fill a prescription for the user, and the like.

In some embodiments, the graphical user interface (UI) of a system disclosed herein is configured to provide information to the user. In further embodiments, the UI is configured to provide information from the user to the system (e.g., via user interaction with the system and/or user providing/uploading images and/or video to the system). Information provided to the user may include, but is not limited to, any of the information disclosed herein including identification (e.g., diagnosis) of the deformity, disorder, and/or injury, treatment for the deformity, disorder, and/or injury, and actions the user may take regarding the deformity, disorder, and/or injury. The system is likewise not limited by the type of information provided by the user to the system. In some embodiments, the graphical UI comprises a touch screen interface.

In some embodiments, the graphical user interface is configured to accept data from the user. In some embodiments, the data from the user comprises selection and/or identification of a body part (e.g., a body part the user believes is deformed, disordered, or injured). In some embodiments, if the user selects a body part the user believes is injured, then the data from the user may also comprise selection of one or more injury symptoms, injury duration, a known injury diagnosis, or a combination thereof. For example, the data from the user may comprise selection of an injured body part (e.g., from a variety of images presented to the user via the UI). The injured body part may be selected from an image of the human body (e.g., an image presented to user (e.g., a diagram of the body or body part) or an image provided by the user (e.g., a picture, 3-D scan, x-ray, CT-scan, ultrasound, etc.) that depicts the injured body part). The injured body part may be selected from a list of body parts. The data from the user may further comprises selection of one or more injury symptoms. For example, the data may further comprise injury symptoms selected from pain, inflammation, deformity, inflexibility, discoloration, and combinations thereof. In a further embodiment, the data may comprise a scale (e.g., from zero to 10) to characterize and/or quantify a symptom.

In some embodiments, the data from the user comprises selection of one or more joint-specific injury symptoms. For example, the joint-specific injury symptoms may be selected from pain, inflammation, deformity, inflexibility, discoloration, and combinations thereof in a toe, ankle, knee, hip, finger, wrist, elbow, shoulder, or neck of the user.

In some embodiments, the data from the user further comprises injury duration.

In some embodiments, the data from the user comprises a known injury diagnosis.

In some embodiments, the output regarding the appropriate medical equipment and/or injury treatment plan is visual output, audio output, physical output, or a combination thereof.

In some embodiments, the system connects to other electronic health systems systems/records/networks. For example, the user account may be linked to the users account at their local physician's office such that the physician has access to the information regarding the data regarding the injury provided by the user, the medical equipment, and/or the treatment plan recommended to the user by the system. As another example, the user account may be linked to the users account at their local physical therapist's office such that the physical therapist has access to the information regarding the injury provided by the user, the medical equipment, and/or the treatment plan recommended to the user by the system. In some embodiments, the system may connect with or be accessible by health insurance agencies (e.g. Medicare, private health insurance). In some embodiments, the system may connect with or be accessible by medical records companies (e.g. EPIC).

Additional objects, features, and advantages of the present disclosure will become apparent from the following detailed description. It should be understood, however, that the detailed description and specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1-FIG. 2 show exemplary methods for a user of a system as disclosed herein to select an injured body part. FIG. 1 shows an exemplary model figure from which a user may select an injured body part. FIG. 2 shows an exemplary “zoomed in” area, from which a user may select a more specific injured body part.

FIG. 3-FIG. 9 show exemplary data that may be provided by the user using a system as disclosed herein to receive an injury diagnosis and/or suitable actions to take regarding the injury. FIG. 3, FIG. 4, and FIG. 5 show exemplary questions regarding injury symptoms that the graphical user interface may pose to a user to be answered on a yes/no basis. FIG. 6 shows an exemplary question regarding injury symptoms (e.g. redness) that the graphical user interface may pose to a user to be answered on a yes/no basis, wherein the questions are accompanied by images to assist the user in answering the question. FIG. 7, FIG. 8, and FIG. 9 show exemplary questions regarding injury symptoms (e.g. swelling) that the graphical user interface may pose to a user, wherein the questions are accompanied by images to assist the user in answering the question. FIG. 7 shows no swelling, FIG. 8 shows mild swelling, and FIG. 9 shows intense swelling. The user may scroll through the images shown in FIG. 7, FIG. 8, and FIG. 9, respectively, in order to select the degree of swelling most representative of the user's symptom.

FIG. 10 shows an exemplary output that the system (e.g. graphical user interface) may provide to the user. The system may apply an algorithm to provide the user with a likely diagnosis (e.g. Runner's Knee). The user interface may additionally provide the user with general details of the likely diagnosis, and suitable information regarding suitable actions for the user to take and/or actions for the user to avoid.

FIG. 11 shows an exemplary output that the system may provide to the user. The output shown in FIG. 11 includes suitable medical equipment (e.g. orthotics, braces), along with a suitable treatment regimen (e.g. at home exercises) for the user to follow. Additionally, FIG. 11 shows that the system may be configured such that the user can upload a picture of the injury every other week in order to track recovery from the injury.

### DETAILED DESCRIPTION

### 1. Definitions

Unless otherwise defined, all technical terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the terms “comprise”, “include”, and linguistic variations thereof denote the presence of recited feature(s), element(s), method step(s), etc. without the exclusion of the presence of additional feature(s), element(s), method step(s), etc.

As used herein, the term “diagnosis” refers to the identification of one or more particular deformities, disorders, or injuries that the user may be experiencing. In some embodiments, providing the user with a “diagnosis” comprises providing the user with an identification of the most likely deformity, disorder, or injury that the user is afflicted with, and additionally providing the user with identification of alternative potential deformities, disorders, or injuries that they may be experiencing. The term “diagnosis” includes a “differential diagnosis”, which refers to identifying the underlying deformity, disorder, or injury that the user is experiencing by comparing the user's symptoms with symptoms known to be associated with one or more conditions having a similar clinical presentation to the symptoms exhibited by the user, in order to identify the underlying deformity, disorder, or injury present in the user.

As used herein, the term “injury” is used in the broadest sense and is meant to encompass any disorder, condition, deformity, or damage to a body part in an individual. In some embodiments, the term “injury” refers to an underlying disorder or condition in the user. In some embodiments, the term “injury” refers to a deformity. In some embodiments, the term “injury” refers to damage to a body part, such as damage incurred as a result of physical trauma to the user.

As used herein, the term “injured” or “injured body part” refers to the location in the body in which the user is experiencing an injury (e.g. disorder, condition, deformity, or damage).

As used herein the term “injury treatment plan” refers to any suitable action for the user to take in order to treat an underlying injury (e.g. disorder, condition, deformity, or damage). Treating an underlying injury refers to steps taken to manage an injury. Steps taken to manage an injury may comprise steps intended to improve one or more symptoms of the injury. For example, “treatment” or an injury (e.g. an “injury treatment plan”) may comprise steps taken to reduce the amount or severity of one or more symptoms of the injury. Treatment of an injury may improve one or more symptoms of the injury. Treatment of an injury may completely eliminate one or more symptoms of the injury.

The term “physical therapy” is used herein in the broadest sense and refers to any therapy provided by a third party to the user focused on rehabilitation/treatment of the injury and/or prevention of a future injury. Physical therapy may be administered to the user by a licensed physical therapist. Alternatively, physical therapy may be administered to the user by a massage therapist, chiropractor, podiatrist, athletic trainer, or other suitable party. The term “athletic trainer” is used herein to refer to a person capable of providing physical therapy to the user from a location such as a high school campus, college campus, or as an affiliate with a sports organization.

The term “physician” as used herein is used in the broadest sense and refers to any person qualified to practice medicine. For example, the term is meant to include a medical doctor, a physician's assistant, a nurse practitioner, and the like.

As used herein, the term “user” refers to an individual. The individual may be the person using the system disclosed herein to guide treatment of an injury. The user may be suffering from an orthopedic or musculoskeletal injury. The user may be any suitable age. For example, the user may be a pediatric user, an adult user, or an elderly user. The user may be any gender.

### 2. Systems and Methods for Self-Guided Treatment

In one aspect, provided herein are systems for self-guided treatment of deformities, disorders, and/or injuries in a user. The systems are configured to accept data from the user, apply an algorithm to the data, and provide output to the user. In some embodiments, the systems comprise a graphical user interface and a processor. In some embodiments, the systems further comprise one or more of a computer-readable code and a memory storage means. In some embodiments, the systems comprise a graphical user interface, a processor, and computer-readable code. In some embodiments, the disclosure provides a system comprising a processor, graphical user interface, a memory storage means, and computer-readable code.

In exemplary embodiments, the graphical user interface comprises a touch screen display. It should be understood, however, that the graphical user interface may comprise other devices, such as computers, which may include one or more other physical means other than a touch screen for the user to interact with the graphical user interface, such as a physical click wheel, a physical keyboard, a mouse and/or a joystick.

In some embodiments, the graphical user interface is configured to receive data regarding a deformity, disorder, and/or injury and transmit the data to the processor. The data may be any suitable data regarding the injury. For example, the user may be suffering from an orthopedic or musculoskeletal deformity, disorder, and/or injury and the data may be suitable data regarding the deformity, disorder, and/or injury. For example, the data may comprise information regarding an injured body part, one or more injury symptoms, injury duration, a known injury diagnosis, or a combination thereof. In any of the embodiments described herein, the user may select an option (e.g. a body part, a symptom, a duration, and the like) provided on the graphical user interface or may manually enter the data themselves. For example, the user may type-in data, such as the location of the injury, duration of the injury, etc. for use by the system.

In some embodiments, the data comprises an injured body part. For example, the data may comprise user selection of an injured body part. The injured body part may be selected from an image of the human body provided on the graphical user interface. For example, the injured body part may be selected from an image of the human body wherein the user can select an area such as the head, neck, shoulder, upper arm, elbow, lower arm, wrist, hand, finger, chest, ribcage, abdomen, spine (e.g. upper back, lower back), hip, buttocks, groin, upper leg (e.g. thigh or hamstring), knee, lower leg (e.g. shin or calf), ankle, heel, foot, or toe. In some embodiments, the injured body part may be selected by first selecting an area of the body as described above and making a subsequent, second selection of a more detailed location within that area. For example, the user may first select an area such as the foot and then the graphical user interface may provide a second, “zoomed in” image of the selected area. The user may make a second selection to show a more specific area of the foot that is injured. Alternatively, the injured body part may be selected from a list of body parts provided on the graphical user interface. In some embodiments, the injured body part is selected from a combination of graphical images of a body parts and a list of body parts.

In some embodiments, the graphical user interface allows a user to identify an injured body part. For example, the graphical user interface, in some embodiments of the system, allows a user to identify an injured body part by selecting the injured body part from an image presented to the user (e.g., an image presented on a monitor of the graphical user interface). In other embodiments, the system allows the user to provide the system with an image or picture of the user's injured body part. For example, the user may upload an image of an injured body part and the system may apply an algorithm to compare the image of the user's body part against a database of similar photographs to select an appropriate diagnosis for the user. Certain conditions would be particularly well suited to such embodiments wherein the user uploads or otherwise provides the system with an image of an injured body part. In particular, suitable conditions include Gout, Bunion, Hammer Toe, Achilles tendonitis midbody and insertional, Achilles tear, Knee effusion, prepatellar bursitis, Knee infection, Scoliosis, Mallet finger, angled finger and toe fractures, Ganglion cyst, CMC joint arthritis, finger arthritis, Gout crystals, Olecranon bursitis, Radial styloid tenosynovitis, biceps tear distal and proximal, AC joint sprain grade II and III, SC joint arthritis, subungual hematoma, and cellulitis.

The disclosure is not limited by the means by which the user provides an image or picture. For example, in some embodiments, the system comprises one or more cameras (e.g., used by the user to take and/or provide an image of the body part). In some embodiments, the image is a three-dimensional image. In some embodiments, the system comprises a scanner (e.g., x-ray scanner) that takes and/or provides an image to the system. Any means known in the art may be used to take and/or provide an image of a user's body part to the system. In some embodiments, the system further comprises a means to access the internet.

In some embodiments, the data further comprises selection of one or more symptoms of the disorder, deformity, and/or injury. For example, the data may further comprise user selection of one or more symptoms selected from pain, inflammation (e.g. swelling), deformity, inflexibility (e.g. stiffness of a joint, muscle inflexibility, diminished movement capability, etc.), discoloration (e.g. bruising, redness), and combinations thereof. In particular embodiments, further details regarding the level of the symptom (e.g. mild, moderate, or severe pain, mild, moderate, or severe swelling, etc.) may be provided by the user. In some embodiments, the user may first select a body part and subsequently select a symptom that the user is experiencing in that particular body part. For example, the user may first select an injured body part (e.g. neck, shoulder, wrist, ankle, etc.) and subsequently select symptoms that the user is experiencing in that area (e.g. pain, inflammation, discoloration, etc.). Alternatively, the user may first select a symptom and subsequently select a body part in which the symptom is occurring. In some embodiments, the user may select symptoms from a list of available symptoms provided on the graphical user interface.

In some embodiments, the data comprises selection of one or more joint-specific symptoms. For example, the data may comprise user selection of one or more of pain, inflammation, deformity, inflexibility, discoloration, and combinations thereof in a toe, ankle, knee, hip, finger, wrist, elbow, shoulder, or neck of the user. In some embodiments, the user may select one or more joint-specific symptoms from a list of symptoms provided on the graphical user interface.

In some embodiments, the user answers a series of “yes” or “no” questions regarding the symptoms that the user is experiencing. For example, the system may ask the user may be asked if they are experiencing pain, inflammation, discoloration, etc. in the injured area and the user may select “yes” or “no” in regard to each symptom. In some embodiments, the system may provide the user with an image and asked whether the user is experiencing similar symptoms as shown in the image. For example, the user may select an injured body part and in subsequent questions, an image of that body part experiencing swelling may be displayed on the graphical user interface (e.g. computer screen). The graphical user interface may display a question to the user in which the user is asked whether they are experiencing swelling as shown in the photo, and the user will select a “yes” or “no” answer. As another example, the user may select an injured body part and an image of that body part experiencing discoloration (e.g. bruising, redness) may be displayed on the graphical user interface. The graphical user interface may display a question to the user in which the user is asked whether they are experiencing discoloration as shown in the photo, and the user may select a “yes” or “no” answer. The above examples may be applied to any injured body part and any suitable symptom for which a photo depiction of the symptom is appropriate.

In some embodiments, the data further comprises information regarding the duration of the injury and/or symptoms. For example, the user may select a duration of the injury from a list of durations provided on the graphical user interface. For example, the duration of the injury may be 1-6 hours, 6-12 hours, 12-24 hours, 2 days, 3 days, 4 days, 5 days, 6 days, 1 week, 2 weeks, 3 weeks, 4 weeks, 5 weeks, 6 weeks, 7 weeks, or more than 2 months. In some embodiments, the data may comprise information about whether or not the user experienced trauma prior to symptom onset. For example, the user may indicate on a yes/no basis whether the user experienced trauma prior to the onset of symptoms.

In some embodiments, the data comprises a known injury diagnosis. For example, the user may manually type in a known injury diagnosis. Alternatively, the user may select a known injury diagnosis from a list of potential injuries provided on the graphical user interface. For example, the user may select an injured body part and subsequently select a known injury diagnosis from a list of potential injuries commonly associated with that body part. The injury may be any orthopedic or musculoskeletal injury. In some embodiments, the known injury is selected from a head injury (e.g. concussion), a neck injury (e.g. TMJ-related pain), a shoulder injury, an elbow injury (e.g. tendonitis, tennis below), a wrist injury (e.g. tendonitis), a hand injury (e.g. osteoarthritis, carpal-tunnel syndrome), a finger injury (e.g. joint trauma, trigger finger, mallet finger), a rib injury, a chest injury, a spinal injury (e.g. thoracic spine pain, lumbar pain, cervical spondylosis, herniated disc, spinal stenosis, lumbago, pars fractures), a hip injury (e.g. bursitis), an abdominal injury, a groin injury, an upper leg injury (e.g. thigh/hamstring injuries), a knee injury (e.g. tendonitis), a lower leg injury (e.g. shin splints), an ankle injury, a heel injury (e.g. Achilles tendonitis), a foot injury (e.g., plantar fasciitis, arch pain, bunions, Lisfranc midfoot injury), or a toe injury (e.g. toe trauma, turf toe, hammer toe). The known injury may a joint injury, a muscle injury, or an injury to one or more ligaments or tendons. For example, the injury may be tendonitis. The injury may be a sprain.

The system is configured to provide output to the user based upon the data received. In some embodiments, the processor is configured to provide output to the user. For example, the processor may be configured to apply an algorithm to the data to provide output to the user. In some embodiments, the computer-readable code is configured to instruct the processor to apply an algorithm to the data to provide output to the user. The system is not limited to any particular output provided to the user. The output provided to the user may comprise information related to the identification of the deformity, disorder, and/or injury. The output provided to the user may comprise information related to a treatment (e.g., a treatment plan) for the deformity, disorder, and/or injury. In some embodiments, the output provides both information related to the identification (e.g., a differential diagnosis) of the deformity, disorder, and/or injury and a recommended treatment plan (e.g., an orthopedic brace, exercise, rest, or combination of same) for the deformity, disorder, and/or injury. In some embodiments, the output to the user comprises information about an alternative user action that the user may take regarding the deformity, disorder, and/or injury. For example, an alternative user action provided to the user may comprise instructions for obtaining a scan (e.g., x-ray, magnetic resonance image (MRI), computed tomography (CT or CAT), ultrasound, or other type of scan) of the deformity, disorder, or injury, instructions to make an appointment with a physician, instructions to call 9-1-1, and/or other instructions regarding care for the deformity, disorder, or injury.

In some embodiments, the output to the user comprises one or more of the following: a diagnosis of the injury; a recommendation of an appropriate medical equipment and/or injury treatment plan for the user; or a recommendation for an alternative user action. In some embodiments, the recommendation for an alternative user action is a recommendation for a diagnostic procedure to be performed on the user.

In some embodiments, the output to the user comprises an injury diagnosis. In some embodiments, the computer-readable code is configured to instruct the processor to apply an algorithm to the data received from the user to diagnose the user with a particular injury. In some embodiments, the computer-readable code is configured to instruct the processor to apply an algorithm to the data received from the user to diagnose the user with a most likely injury, and to provide a list of other potential injuries that the user may be indicating based upon the data provided to the system. In some embodiments, the most likely injury and/or list of other potential injuries is accompanied by additional information about the injury. For example, the most likely injury and/or list of other potential injuries may be presented on the graphical user interface along with information regarding common causes of the injury, common symptoms of the injury, ways to avoid similar injuries in the future, suitable treatments for the injury, etc. This information may be provided in an easily digestible means for the user, such as a bullet point list.

In some embodiments, the output comprises a recommendation of an appropriate medical equipment and/or injury treatment plan for the user. In some embodiments, the computer-readable code is configured to instruct the processor to apply an algorithm to the data received from the user to determine the appropriate medical equipment and/or injury treatment plan for the user. In some embodiments, the output provided by the system to the user comprises both a likely injury diagnosis (or list of likely injuries) and a recommendation for appropriate medical equipment and/or an injury treatment plan suited to manage the injury.

In some embodiments, the injury treatment plan comprises an at home treatment regimen (e.g. icing, heating, medications, rehabilitation exercises), a visit to a physician, physical therapy sessions, or a combination thereof. In some embodiments, the injury treatment plan is provided in written form (e.g. as a bullet point list, paragraph, etc.). In some embodiments, the injury treatment plan is provided in video format. For example, the injury treatment plan may be rehabilitation exercises, which are exemplified in video format. For example, a written injury treatment plan or suitable videos may be displayed directly on the graphical user interface. Alternatively or in addition, a written injury treatment plan or suitable videos may be emailed to the user.

In some embodiments, the injury treatment plan comprises medications to be used by the injured user. For example, suitable medications for an at home treatment regimen include pain medications and/or anti-inflammatory medications. Such medications may be over-the-counter medications. Anti-inflammatory medications may be non-steroidal anti-inflammatory drugs (NSAIDS) such as ibuprofen, aspirin, naproxen, and the like. In some embodiments, medications are taken orally. In some embodiments, medications may be applied topically. For example, an at home treatment regimen may comprise instructing the user to apply a topical NSAID (e.g. Voltaren) to the injured area. In some embodiments, the system may recommend medications and send them directly to the user. For example, the system may send over-the-counter medications directly to the user. In some embodiments, medications may require a prescription, which may be provided by the system or a third party (e.g. physician) using the system. For example, a physician with access to a user's account on the system may be able to provide the user with a prescription for a medication as needed. In some embodiments, the prescription may be sent to a pharmacy selected by the user to be filled by the pharmacy.

In some embodiments, the graphical user interface is further configured to provide output regarding the appropriate medical equipment and/or injury treatment plan to the user. For example, the output may be visual output, audio output, physical output (e.g. an actual brace or medical device for the user to try on), or a combination thereof. In some embodiments, a system is provided comprising the computer-readable code, user interface, user input, and output, where the output is provided via a secure link (e.g., shown on the user interface that the user accesses via the internet).

In some embodiments, the algorithm is a decision-tree algorithm. For example, the processor may apply a decision-tree algorithm to determine the next steps in the algorithm based upon each subsequent data provided by the user in order to arrive at a final determination of the appropriate medical equipment and/or injury treatment plan for the user. In some embodiments, the system is configured such that an indication of an emergency condition (e.g. head injury, blood clot, etc.) provides the user with a recommendation to go to a suitable clinic (e.g. urgent care facility) to receive immediate diagnosis and appropriate treatment.

The system may maintain a database of service providers (e.g. physicians, clinics, urgent care facilities, emergency rooms, etc.) that may be accessed by the system to determine the appropriate recommendation for the user. In some embodiments, the system may additionally maintain information regarding diagnostic capabilities of the service providers (e.g. MRI capability, X-ray capability, etc.) to ensure that the service provider recommended to the user is capable of performing the necessary diagnostic procedure and/or treatment. For example, if the user or the data provided by the user indicates that the user is potentially suffering a head injury (e.g. by user selection of the head or by user input of a known head injury), the decision tree algorithm may cause the system to provide the user with a recommendation to visit a service provider with the capability to confirm the head injury and provide the appropriate treatment. As another example, if the user or the data provided by the user indicates that the user if potentially suffering from a blood clot, the decision tree algorithm may cause the system the provide the user with a recommendation to visit a service provider with the capability to confirm that a blood clot is present. The system may additionally provide information regarding the clinic location, hours, etc.

In some embodiments, the output regarding the appropriate medical equipment and/or injury treatment plan to the user is visual output. For example, the output may be an image of the appropriate medical equipment, a description of the appropriate medical equipment (e.g. the name of the medical equipment, the purpose of the appropriate medical equipment, sizing options for the equipment, the cost of the medical equipment), a video demonstrating appropriate application of the medical equipment (e.g. video showing appropriate way to wear a brace, appropriate fit of a brace, etc.) locations to purchase the appropriate medical equipment, a description of an at home treatment regimen (e.g. icing, heating, stretching, medications, rehabilitation exercises, etc.), a recommendation for physician visit, recommendation for physical therapy sessions, or a combination thereof. In some embodiments, the output may comprise an e-mail to the user. For example, the output may be an e-mail to the user containing information containing the diagnosis, an at home treatment regimen, a description of the appropriate medical equipment, etc. In some embodiments, the output may comprise an e-mail sent to the user on the same day as the user interacting with the system for self-guided injury treatment.

In some embodiments, the output regarding the appropriate medical equipment is a physical piece of medical equipment for the user to try. For example, the system may further comprise a means for providing medical equipment to the user. For example, the system may further comprise a robotic vending machine containing medical equipment. The system may determine the appropriate medical equipment for the user based upon the data provided by the user and provide the appropriate medical equipment through the robotic vending machine. Alternatively, the system may determine the appropriate medical equipment for the user, provide visual output regarding the appropriate medical equipment (e.g. a picture of the equipment, the name of the equipment, etc.), and allow for the user to select the appropriate equipment through the robotic vending machine. The pieces of equipment could be displayed to the user to allow visual inspection of the equipment prior to selection. For example, the pieces of equipment could be shown in an actual display case within the robotic vending machine. Alternatively, the pieces of equipment could be displayed virtually on the screen of the graphical user interface. In some embodiments, the output may be a sample medical equipment for the user to try on, but not purchase. For example, the output may be a sample medical equipment (e.g. a brace) that is provided to the user (such as through the robotic vending machine) on a retractable cord for the user to try on. The retractable cord would allow the machine to re-claim the medical equipment after the user has tried it on. For example, this would allow the user to try on multiple sizes of equipment to select the appropriate size prior to purchasing.

In some embodiments, the output regarding the appropriate medical equipment is a recommendation for a suitable medical equipment (e.g. brace) along with a means for the user to purchase the recommended medical equipment. For example, the output may be an image of a suitable medical equipment along with a link for the user to purchase the medical equipment. In some embodiments, the user may receive a discount on medical equipment purchased within a set time frame after the output regarding the appropriate medical equipment is provided. For example, the user may receive a discount on the recommended medical equipment if purchased within 24 hours of injury diagnosis/equipment recommendation using the system described herein.

The appropriate medical equipment may be any suitable medical equipment for the symptoms/injury of the user. For example, appropriate medical equipment may include one or more helmets (e.g. cycling helmets, football helmets, horse-back riding helmets) headbands (e.g. foam/gel headbands for soccer, basketball, lacrosse, field hockey, etc.), glasses (e.g. vision protection goggles, blue-light blocking glasses for computer use), cervical traction devices, collars (e.g. soft cervical collars), wraps (e.g. neck wraps, shoulder wraps), braces (e.g. posture support braces, hinged knee braces, patellar stabilizer braces, ankle braces, CMC osteoarthritis braces), corsets (e.g. lumbar corsets), belts (e.g. SI belts), hip protectors, sleeves (e.g. knee sleeves, knee sleeves with patellar cutouts, neoprene ankle sleeves, neoprene elbow sleeves), straps (e.g. patellar tendon straps, hammer toe straps, elbow compression straps), boots (e.g. CAM walker boots), shoes, heel lifts, heel wedges, orthotics, splints (e.g. night splints, aluminum splints), spacers (e.g. bunion spacers), stockings (e.g. compression stockings), head pads/dancers, slings (e.g. shoulder slings), gloves (e.g. compression gloves), TENS units, bath devices, home exercise equipment (e.g. pedaling devices), and rehabilitation products (e.g. foam rollers, Thera-bands, etc.). In some embodiments, more than one medical equipment may be recommended. In some embodiments, a combination of one or more medical equipment pieces and an injury treatment plan may be recommended. For example, the algorithm may determine that one or more medical equipment pieces, one or more at home exercises, one or more at home treatment regimens, physical therapy sessions, and/or a physician's visit is needed by the user.

In some embodiments, the system is further configured to receive background information from the user. For example, the user may provide background information including their name, e-mail address, mailing address, phone number, height, weight, age, and gender. Accordingly, in some embodiments, a system is provided comprising memory/data storage device (e.g., local and/or remote (e.g. accessed via the internet)). In some embodiments, the background information may be stored in the system and used as part of the algorithm to provide the user with an appropriate medical equipment recommendation. For example, background information including gender, age, weight, height, etc. may be informative for the algorithm to determine the likely injury diagnosis, other potential injuries, suitable medical equipment, suitable treatment procedures, and/or suitable sizes for medical equipment (e.g. braces) recommended to the user.

In some embodiments, the user may sign into an account using the systems described herein. The account may be an account for a single user or for multiple users (such as a family account). The system may comprise a memory/data storage component configured to store any suitable information affiliated with one or more users. For example, the system may comprise a memory component configured to store background information provided by the user and any suitable information regarding the injury, including symptoms, images, test results, treatments, recovery/progress, user notes, input provided by a third party service provider, etc. For example, the system may comprise a memory component configured to store background information provided by each user, input data regarding the injured body area and/or symptoms the user is experiencing, output data regarding the likely injury diagnosis (or list of potential injuries) along with suitable medical equipment, at home exercises, etc. for each user, and any additional data related to the injury or recovery therefrom, including data provided by the user and data provided by a third party service provider. In some embodiments, the accounts for each user on a family account are protected, such that the single user is only able to access their specific account and not the information for other users on the family account. In some embodiments, the accounts for each user on a family account are linked, such that each user is able to see the accounts of other users. Linked accounts may be preferable in instances where, for example, a parent requires access to a child's account to manage a child's injury. The user (or parent) may provide their personal information or may remain anonymous.

In some embodiments, the account is linked to an external data storage system (e.g. a cloud-based system) such that the user information, data, history of medical equipment recommendations, input from a service provider such as a physician, physical therapist, athletic trainer, etc. is maintained. The system may access this information when the user logs in to their account to use the system for a recommendation of a medical equipment or injury treatment plan. Multiple user accounts (e.g. a family account) may be linked on the same external data storage system (e.g. cloud-based system).

In some embodiments, the system may be placed in a location that also has medical equipment on-hand. For example, the system may be located in a pharmacy or a clinic that additionally sells the medical equipment, home exercise equipment, or rehabilitation products that the user may be recommended to use by the system. Alternatively, the system may be able to provide links, instructions, etc. for the user to order the necessary medical equipment for delivery to a separate location. For example, the system may allow the user to provide their address and purchase the necessary medical equipment from an external source (e.g. from a suitable internet website) to be shipped to the user's home.

In some embodiments, the system has a physical location, such as a kiosk, at a suitable location (e.g. store, pharmacy, clinic. etc.) In other embodiments, the system may be downloaded as an application for use on personal devices (e.g. mobile devices, tablets, etc.).

In some embodiments, the system connects to other electronic health systems systems/records/networks. For example, the user account may be linked to the users account at their local physician's office such that the physician has access to the information regarding the data regarding the injury provided by the user, the medical equipment, and/or the treatment plan recommended to the user by the system. As another example, the user account may be linked to the users account at their local physical therapist's office such that the physical therapist has access to the information regarding the injury provided by the user, the medical equipment, and/or the treatment plan recommended to the user by the system.

In some embodiments, the output regarding an injury treatment plan is a recommendation for a visit to a third party. For example, the output may provide a direct referral to a local third party, such as a physician, a clinic to perform a diagnostic imaging procedure, or a suitable provider to administer physical therapy to the user (e.g. a physical therapist, a massage therapist, a chiropractor, etc.). In some embodiments, the system maintains a database of service providers. In some embodiments, the system is configured to search the database for a list of service providers within a given radius of the zip code of the user. The system may apply the user input regarding the injury to search the database for a suitable list of providers to address the particular injury or condition the subject appears to be afflicted with. For example, the system may apply an algorithm to the user input to diagnose the user with a potential knee injury, and provide the user with a list of orthopedic doctors specializing in knee injury treatments. As another example, the system may apply the user input to diagnose the user with a potential hip injury, and provide the user with a list of orthopedic hip specialists.

In some embodiments, the system provides the user with a direct referral or a list of local third parties that accept the health insurance of the user. Such an output would allow for the user to receive a direct referral to a suitable local third party without the need to see a primary care physician beforehand. For example, the output may be a recommendation for a physician's office (e.g. orthopedic clinic, rheumatology clinic, etc.) and/or a physical therapy session (e.g. visit to a physical therapist, athletic trainer, podiatrist, chiropractor, massage therapist, etc.) In some embodiments, the system may comprise links displayed on the graphical user interface to direct the user to the option to connect with a local physician for a follow-up appointment regarding the injury. For example, the system may comprise links displayed on the graphical user interface to direct the user to local physicians offering follow up appointments on the same day, the next day, within two days, within three days, within four days, within five days, within one week, etc. In some embodiments, the system may comprise links display on the graphical user interface to direct the user to connect with a local third party for physical therapy sessions. For example, the system may comprise links displayed on the graphical user interface to direct the user to local physical therapists, massage therapists, chiropractors, podiatrists, athletic trainers, or other entities capable of providing physical therapy. The links may direct the user to a suitable party offering follow up appointments on the same day, the next day, within two days, within three days, within four days, within five days, within one week, etc.

In some embodiments, the system provides an option of interacting with a live health care provider. For example, the system may provide an option for a telephone conference or a virtual visit (e.g. with a live camera) with a health care provider such that the user can explain their symptoms or diagnosis, and the health care provider can verify or provide the appropriate brace selection and other health recommendations (e.g. at home treatment plan). In such embodiments, the system may further comprise a camera to allow for a virtual visit with the health care provider. In some embodiments, the system may further comprise a camera such that the user can take pictures of the affected body part. Alternatively, the system may allow the user to upload a previously taken picture of the affected body part and/or enter the dimensions of the affected body part (e.g. circumference of the wrist, elbow, knee, etc.). The system may use the picture taken/provided by the user and/or the dimensions of the affected body part to predict the correct size of the recommended product.

In some embodiments, the output provided by the system to the user is a recommendation for an alternative user action. For example, an alternative user action provided to the user may comprise instructions for obtaining a diagnostic procedure, such as an X-ray, magnetic resonance image (MRI), computed tomography (CT or CAT), ultrasound, bone scan, electromyogram (EMG), or other type of scan of the deformity, disorder, or injury. As another example, an alternative user action may comprise instructions to make an appointment with a physician, instructions to call 9-1-1, and/or other instructions regarding care for the deformity, disorder, or injury.

In particular embodiments, the recommendation for an alternative user action comprises a recommendation for a diagnostic procedure. In some embodiments, the system is configured to recommend that the user receive a diagnostic procedure to confirm a likely injury diagnosis. In such embodiments, the output provided by the system may comprise both a likely injury diagnosis (and optionally alternative possible injuries) and a recommendation for a diagnostic procedure. For example, the system may be configured to recommend that the user receive a diagnostic imaging procedure, such as an magnetic resonance imaging (MRI) scan, a computerized tomography (CT) scan, X-ray, ultrasound, or bone scan. As another example, the system may recommend that the user receive an electromyogram. For example, the algorithm may be configured to determine whether a diagnostic procedure is recommended based upon the user background information, injury, and/or injury symptoms. In such embodiments, the system may be configured to provide the user with an order to obtain a diagnostic procedure. The order may be legitimized in such a way that a local service provider is able to perform the diagnostic procedure without the need for intervening steps, such as physician visits prior to the diagnostic procedure being performed. For example, the system may provide the user with an order to obtain a diagnostic procedure. In some embodiments, the user may provide the order to a service provider of their choice. In some embodiments, the system may send the order directly to a service provider. As described above, the system may maintain a database of service providers (e.g. physicians, clinics, urgent care facilities, emergency rooms, etc.) that may be accessed by the system to determine the appropriate recommendation for the user. In some embodiments, the system may additionally maintain information regarding diagnostic capabilities of the service providers (e.g. MRI capability, X-ray capability, etc.) to ensure that the service provider recommended to the user is capable of performing the necessary diagnostic procedure and/or treatment. The service provider may perform the diagnostic procedure on the user without the need for a recommendation for the diagnostic procedure from a physician.

In some embodiments, the system is configured to recommend that the user receive a diagnostic procedure and to further recommend one or more local providers capable of administering the diagnostic procedure to the user. For example, the graphical display may provide the user with a recommendation for a specific diagnostic procedure (e.g. MRI, CT scan, X-ray, ultrasound, EMG), and may further provider one or more providers based upon an zip code provided by the user (e.g. an zip code provided by the user as background information).

In some embodiments, the system may further comprise a means for the user and/or local provider to upload the results of the diagnostic procedure. For example, the system may comprise a means for the user and/or local provider (e.g. radiologist) to upload the results of an MRI scan, CT scan, X-ray, bone scan, ultrasound, etc.). The algorithm may be configured to apply the results of the diagnostic procedure to provide the user with an injury diagnosis. In some embodiments, the system may comprise a means for the user and/or local provider to upload a copy of the diagnostic image itself (e.g. a copy of the X-ray image, a copy of the MRI image, etc.). Alternatively or in addition, the system may comprise a means for the user and/or local provider to upload the local provider's assessment of the diagnostic procedure. For example, the local provider may assess the diagnostic image and determine a diagnosis (e.g. fractured bone, torn ligament, muscle sprain, etc.) based upon the image. This diagnosis may be entered into the system (e.g. entered as a known diagnosis as part of the data provided to the system) and the algorithm may be configured to apply this diagnosis to recommend a suitable medical equipment and/or treatment regimen for the user based upon the known diagnosis. For example, if an X-ray indicates that the user has a bone fracture, the user and/or local provider may upload this diagnosis into the system and the system may apply the diagnosis to determine the appropriate recommendation for medical equipment and/or injury treatment plan for the user.

In some embodiments, the injury treatment plan for the user may comprise attending one or more physical therapy sessions. The physical therapy session may be provided by a local physical therapist, massage therapist, chiropractor, podiatrist, athletic trainer, or other entities capable of providing physical therapy. In such embodiments, the system may be configured to provide the user a list of local third parties (e.g. physical therapists) which may administer the physical therapy session(s) to the user. For example, the system may be configured to provide the user with a list of local third parties based upon an zip code provided by the user (e.g. an zip code provided as part of the background information provided by the user). In some embodiments, the system may be configured to allow the user to select a local high school or college campus to see local athletic trainers that may be available to administer the physical therapy session(s) to the user.

In some embodiments, the system will have an option for service providers to subscribe to a preferred providers list. For example, service providers such as physical therapists, orthopedic clinics, athletic trainers, physicians available for a virtual visit, and the like may subscribe to a preferred providers list on the system such that the system will recommend the service provider to users within a designated distance of the service provider (e.g. within 50 miles, within 25 miles, etc.)

In some embodiments, the system is configured to be accessible by one or more service providers. In some embodiments, the memory component stores additional data provided to the system by each of the one or more service providers. For example, the system may be configured to allow service providers including physicians (e.g. medical doctors, nurse practitioners, physician's assistants) physical therapists, athletic trainers, and the like to access the user's account on the system. The service providers may provide data to the system, including test results (e.g. results from a diagnostic imaging procedure), notes regarding recovery from the injury or other symptoms the user appears to be experiencing, information regarding the injury treatment plan (e.g. updates to an at home treatment regimen including exercises, stretching, medications, etc.), or additional information pertinent to the injury. The service provider may be licensed to practice in the state in which the user resides. For example, the user may grant the service provider access to the user's account such that the service provider can provide feedback to the user regarding the injury, treatment, recovery process, etc. In some embodiments, the user may grant a service provider such as a physical therapist access to the user's account on the system such that the service provider can provide the user feedback regarding particular exercises that may be beneficial, additional at home treatment regimens that may be beneficial to the user, updates regarding how the user is progressing in recovering from the injury, or additional information that may be helpful to the user in the injury recovery process. Accordingly, provided herein is a comprehensive system that effectively represents a virtual orthopedic office visit that provides a user with the ability to receive a likely injury diagnosis, obtain information regarding the injury, be recommended suitable medical equipment and/or treatment regimens to be followed to manage the injury, obtain a diagnostic procedure if necessary, obtain information regarding local service providers (e.g. physical therapists, physicians, athletic trainers, etc.), and receive feedback from the local service provider regarding the injury and recovery therefrom. In some embodiments, the system may be configured to allow the user to input additional information into the system regarding the injury and recovery therefrom. For example, the user may update their information regarding injury symptoms (e.g. pain, swelling, redness, etc.) over the course of treatment to track their recovery from the injury.

In some embodiments, the system may be configured to coordinate with other agencies. For example, the system may be further configured to coordinate with health insurance companies (e.g. Medicare, private health insurance agencies) such that the user may grant the health insurance company access to their records. Such embodiments may be preferable in instances wherein the system recommends that the user obtain a diagnostic procedure (e.g. MRI, CT scan, etc.). In such embodiments, the user could grant their health insurance company access to their account/records (e.g., the records stored in a cloud-based system as described herein) such that the health insurance agency can pay for the required diagnostic procedure. In some embodiments, the system coordinates with medical records agencies (e.g. EPIC) such that user records on the system are available to other service providers (e.g. physicians, physical therapists, athletic trainers), with the permission of the user. All third party access to user data will be protected in accordance with the Health Insurance Portability and Accountability Act of 1996 (HIPAA), such that the user must willingly grant access to the third party.

1. A system for self-guided injury treatment, comprising:
a. a graphical user interface; and
b. a processor;
wherein the graphical user interface is configured to receive data regarding an injury from a user and transmit the data to the processor; and
wherein the processor is configured to apply an algorithm to the data to provide output to the user on the graphical user interface,
wherein the output provided to the user comprises one or more of:
i. a diagnosis of the injury;
ii. a recommendation of an appropriate medical equipment and/or injury treatment plan for the user; and
iii. a recommendation for an alternative user action.

2. The system of claim 1, wherein the graphical user interface comprises a touch screen interface.

3. The system of claim 1, wherein the data from the user comprises selection of an injured body part, one or more injury symptoms, injury duration, a known injury diagnosis, an image of an injured body part, or a combination thereof.

4. The system of claim 3, wherein the data from the user comprises selection of an injured body part.

5. The system of claim 4, wherein the user selects an injured body part from an image of the human body or from a list of body parts.

6. The system of claim 4, wherein the data from the user further comprises selection of one or more injury symptoms selected from pain, inflammation, deformity, inflexibility, discoloration, and combinations thereof.

7. The system of claim 3, wherein the data from the user comprises selection of one or more joint-specific injury symptoms selected from pain, inflammation, deformity, inflexibility, discoloration, and combinations thereof in a toe, ankle, knee, hip, finger, wrist, elbow, shoulder, or neck of the user.

8. The system of claim 3, wherein the data from the user further comprises injury duration.

9. The system of claim 3, wherein the data from the user comprises a known injury diagnosis.

10. The system of claim 1, wherein the injury treatment plan comprises an at home treatment regimen, a visit to a physician, physical therapy sessions, or a combination thereof.

11. The system of claim 1, wherein the appropriate medical equipment is selected from helmets, headbands, glasses, cervical traction devices, collars, wraps, braces, corsets, belts, hip protectors, sleeves, straps, boots, shoes, heel lifts, heel wedges, orthotics, splints, spacers, stockings, head pads/dancers, slings, gloves, transcutaneous electrical nerve stimulation (TENS) units, bath devices, home exercise equipment, and rehabilitation products.

12. The system of claim 1, wherein the output regarding the appropriate medical equipment and/or injury treatment plan comprises an image of the appropriate medical equipment, a video demonstrating the proper fit and/or use of the medical equipment, locations to purchase the appropriate medical equipment, a description of an at home treatment regimen, a recommendation for physician visit, a recommendation for physical therapy sessions, or a combination thereof.

13. The system of claim 1, wherein the system is further configured to receive background information from the user, wherein the background information comprises the user's name, e-mail address, mailing address, phone number, height, weight, age, gender, or a combination thereof.

14. The system of claim 1, wherein the recommendation for an alternative user action comprises a recommendation that the user receive a diagnostic procedure selected from a magnetic resonance imaging (MRI) scan, a computerized tomography (CT) scan, an X-ray, an ultrasound, and an electromyogram (EMG).

15. The system of claim 1, wherein the system further comprises a database of service providers, wherein the system is configured to apply the data provided by the user recommend a local service provider or a list of local service providers to the user.

16. The system of claim 1, further comprising a memory component.

17. The system of claim 1, further comprising a computer-readable code, wherein the computer-code is configured to instruct the processor to apply the algorithm to the data to provide the output to the user on the graphical user interface.

18. The system of claim 17, wherein the system is configured to be accessible by one or more users, wherein the memory component stores the data regarding the injury for each of the one or more users and stores the output provided by the system to each of the one or more users.

19. The system of claim 19, wherein the system is configured to be accessible by one or more service providers, wherein the memory component stores additional data provided to the system by each of the one or more service providers.

20. A system for self-guided injury treatment, comprising:
a. a graphical user interface;
b. a processor;
c. a memory component; and
d. a computer-readable code,
wherein the graphical user interface is configured to receive data regarding an injury from a user and transmit the data to the processor,
wherein the computer-readable code is configured to instruct the processor to apply an algorithm to the data to provide output to the user on the graphical user interface,
wherein the output provided to the user comprises one or more of:
i. a diagnosis of the injury;
ii. a recommendation of an appropriate medical equipment and/or injury treatment plan for the user; and
iii. a recommendation for an alternative user action, and
wherein the memory component stores the data regarding the injury provided to the system by the user.
